(19) United States
(12) Patent Application Publication
(10) Pub. No.: US 2021/0142342 A1
(43) Pub. Date: May 13, 2021

(54) METHODS AND SYSTEMS FOR PROVISIONING DIGITAL DATA TO AN INDIVIDUAL

(71) Applicant: Zhiqing Cheng, Dayton, OH (US)

(72) Inventor: Zhiqing Cheng, Dayton, OH (US)

(21) Appl. No.: 17/094,321

(22) Filed: Nov. 10, 2020

Related U.S. Application Data
(60) Provisional application No. 62/933,481, filed on Nov. 10, 2019.

(51) Int. Cl. G06Q 30/02; G16H 30/40; G16H 50/50; G16H 50/20; G06F 16/22; G06F 21/60; G06T 11/00; G06T 17/20; A61B 34/10

(52) U.S. Cl. CPC G06Q 30/0201; G16H 30/40; G16H 50/50; G16H 50/20; G16H 20/40; G06F 21/602; G06T 11/003; G06T 17/20; A61B 34/10; G06F 16/22

(57) ABSTRACT

Disclosed herein is a system for provisioning digital data of an individual, in accordance with some embodiments. Accordingly, the system may include a communication device configured for receiving a request from a business provider device associated with a business provider. Further, the communication device may be configured for transmitting a data block of a digital twin data to the business provider device. Further, the system may include a processing device communicatively coupled to the communication device. Further, the processing device may be configured for processing the request. Further, the processing device may be configured for extracting the data block based on the processing. Further, the system may include a storage device communicatively coupled to the processing device. Further, the storage device may be configured for retrieving the data block.

The current application claims a priority to the U.S. Provisional Patent application Ser. No. 62/933,481 filed on Nov. 10, 2019.

### FIELD OF THE INVENTION

Generally, the present disclosure relates to the field of data processing. More specifically, the present disclosure relates to methods and systems for provisioning digital data of an individual.

### BACKGROUND OF THE INVENTION

Existing techniques for provisioning digital data of an individual are deficient with regard to several aspects. For instance, current technologies do not describe and represent multiple human characteristics (such as performance, physical attributes, behavior) associated with the individual in a unified form. Furthermore, current technologies lack bio-fidelity and scientific truth in describing human characteristics.

Therefore, there is a need for improved methods and systems for provisioning digital data to the individual that may overcome one or more of the above-mentioned problems and/or limitations.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

Disclosed herein is a system for provisioning digital data of an individual, in accordance with some embodiments. Accordingly, the system may include a communication device configured for receiving a request from a business provider device associated with a business provider. Further, the request may include an identifier associated with the individual and at least one key associated with at least one data block of a digital twin data associated with the individual. Further, the digital twin data may include structural data representing at least one structure of the individual, at least one functional data representing at least one function of the individual, and behavioral data representing at least one behavior of the individual. Further, the communication device may be configured for transmitting the at least one data block of the digital twin data to the business provider device. Further, the business provider device may be configured to use the at least one data block to personalize at least one of a product and a service provisioned to the individual. Further, the system may include a processing device communicatively coupled to the communication device. Further, the processing device may be configured for processing the request. Further, the processing device may be configured for extracting the at least one data block based on the processing. Further, the system may include a storage device communicatively coupled to the processing device. Further, the storage device may be configured for retrieving the at least one data block.

Further disclosed herein is a method for provisioning digital data of an individual, in accordance with some embodiments. Accordingly, the method may include receiving, using a communication device, a request from a business provider device associated with a business provider. Further, the request may include an identifier associated with the individual and at least one key associated with at least one data block of a digital twin data associated with the individual. Further, the digital twin data may include structural data representing at least one structure of the individual, at least one functional data representing at least one function of the individual, and behavioral data representing at least one behavior of the individual. Further, the method may include processing, using a processing device, the request. Further, the method may include retrieving, using a storage device, the at least one data block. Further, the method may include extracting, using the processing device, the at least one data block based on the processing. Further, the method may include transmitting, using the communication device, the at least one data block of the digital twin data to the business provider device. Further, the business provider device may be configured to use the at least one data block to personalize at least one of a product and a service provisioned to the individual.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform consistent with various embodiments of the present disclosure.

FIG. 2 is a block diagram of a system for provisioning digital data of an individual, in accordance with some embodiments.

FIG. 3 is a block diagram of the system for provisioning the digital data of the individual, in accordance with some embodiments.

FIG. 4 is a flowchart of a method for provisioning digital data of an individual, in accordance with some embodiments.

FIG. 5 is a flowchart of a method for generating the digital twin data, in accordance with some embodiments.

FIG. 6 is a flowchart of a method for updating the digital twin data, in accordance with some embodiments.

FIG. 7 is a flowchart of a method for individualizing the template model data, in accordance with some embodiments.

FIG. 8 is a flowchart of a method for individualizing the template model data, in accordance with some embodiments.

FIG. 9 is a flowchart of a method for individualizing the template model data, in accordance with some embodiments.

FIG. 10 is a flow diagram of a method for facilitating the generating of a human digital twin, in accordance with some embodiments.

FIG. 11 is a flow diagram of a method for facilitating the unification of model structures, in accordance with some embodiments.

FIG. 12 is a flow diagram of a Human Digital Twin (HDT) data management system, in accordance with some embodiments.

FIG. 13 is a block diagram showing an HDT vendor, a human individual, a business, and a Human Digital Twin (HDT), in accordance with some embodiments.

FIG. 14 is a block diagram of a computing device for implementing the methods disclosed herein, in accordance with some embodiments.

### DETAIL DESCRIPTIONS OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of methods and systems for provisioning digital data to an individual, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor and at least one actuator. Examples of the one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, public database, a private database and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on.

Further, one or more steps of the method disclosed herein may be initiated, maintained, controlled and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g. username, password, passphrase, PIN, secret question, secret answer etc.) and/or possession of a machine readable secret data (e.g. encryption key, decryption key, bar codes, etc.) and/or or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera and so on. Likewise, the one or more steps may include receiving, using the communication device, the one or more embodied characteristics from one or more biometric sensors.

Further, one or more steps of the method may be automatically initiated, maintained and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data and any intermediate data therebetween corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

Overview:

The present disclosure describes methods and systems for provisioning digital data of an individual. Further, every human may be different as each is unique and different from others. Further, the disclosed system creates individualized models for each person so that personal uniqueness is well described. Creating a digital human model for each person requires extensive work. To keep the effort tractable and affordable, the disclosed system uses standard models as the templates and then create individualized (instance) models from the template models via fitting, morphing, and scaling based on the personal data. Further, a Human Digital Twin (HDT) generated with the disclosed system provides different levels of details and fidelity of an individual human body.

Further, the HDT may be based on personal data and physics/first principles. Further, the HDT may be utilized in various human-centered applications, such as personalized products, services, training, education, and performance enhancement.

Further, the HDT may be used in spine surgery. The HDT of a patient can be used to simulate the effects of surgical options before the operation. Surgeons may use the results of the simulation to determine whether a specific surgery is right for the patient and what effects will have on the forces developed in the rest of the spine. Surgical device companies can also use the HDT with 3D printing to make patient-specific implants.

Further, the HDT may be used in exoskeletons. Further, the exoskeleton realizes its performance through intimate contact and interaction with a human user. The overall performance depends on many factors including the degree of coupling to the human. Further, the HDT with its human shape model and musculoskeletal model can be used to investigate numerous “what-if” design scenarios at a relatively low cost. This includes studying the effects of variability, determining the “best” geometries for performance, and to examine the linkage between form and function using virtual experimentation, to name a few.

Further, the HDT may be used for rehabilitation. Further, stroke is one of the leading causes of disability around the world. Whenever a stroke happens, the stroke survivor's brain commands cannot reach some muscles although those muscles could contract. Therefore, their body cannot perform the expected motion, thus hampering their activities of daily living. The objective of rehabilitation is to externally drive the human body to move to improve muscle movements through robotic devices. This human-robot interaction is a critical factor in the design of a successful robotic rehabilitation device. The HDT of a patient can be used to customize the design of the device or to adjust the setting of the device so that the effective human-robot interaction can be achieved to improve rehabilitation.

Further, the HDT may be used in a prosthesis. Further, the prosthesis refers to an artificial substitute or replacement of a part of the body. Prostheses for joints are the hip, knee, elbow, ankle, and finger joints. Prosthetic designs need to be personalized to accommodate the different needs of different customers. From the HDT, the human shape model can be used in 3D printing to create the replacement which just fits, a musculoskeletal model can be used to simulate and predict biomechanical effects of replacement on normal movement, and a full-body FE model can predict the stress in the joint being replaced.

Further, the HDT may be used in a personalized safety system in vehicles. Human injury risks are highly affected by the occupant size and shape, and material properties of human biological tissues. Therefore, the full-body finite element model from the HDT of a specific occupant can greatly enhance the understanding of how to design a safety system to protect the occupant better. It can be imagined that future vehicle manufactures can provide a specific set of safety configuration parameters based on the digital twin of a customer. When the customer is using the vehicle, the safety system can rapidly pre-set the configuration parameters to optimize the protection of the occupants in motor vehicle crashes. This personalized safety design concept can also be applied to other safety systems interacting with a human, such as helmets, sports shoes, etc.

Further, the HDT may be useful to remote medicine and tele healthcare. Further, the HDT, with a higher level of details and bio-fidelity derived from personal MRI, CT, or other forms of medical images, can be used in remote surgery or the augmented reality assisted surgery (ARAS).

Further, the HDTs may become valuable assets for both individuals and businesses. For an individual, having his digital twin can substantially improve the quality of life with personalized products and personalized services that best fit his needs. For a business, by utilizing the data provided by the HDT, the business will be able to create products and services to best fit an individual customer, adding values to the products and services thus differentiating from others.

Further, the disclosed system may be configured for generating invariants of HDT, that may include a Warfighter Digital Twin (WDT), but are not limited to, Firefighter Digital Twin (FDT), an Astronaut Digital Twin (ADT), and any other type of HDT that is specifically constructed for a specific group of people who share common features/characteristic, conditions, and requirements. Further, the WDT may include a special HDT specifically constructed for warfighters. Further, the FDT may include a special HDT specifically constructed for firefighters. Further, the ADT may include a special HDT specifically constructed for astronauts.

Further, the Human Digital Twin (HDT) may include a digital replica of an individual human. Further, the HDT digitally represents a multitude of features, attributes, characteristics, performance, and behavior of an individual human based on the personal data and physics/first principles. The HDT may include the individualized and unified digital models that may provide a compact, parameterized representation of an individual human and the data containers of personal physical, biological, and physiological data. The HDT may be used in human-centered applications including personalized products and services. Further, the HDT may include individualized and unified digital models that provide compact, parameterized representation of an individual human and data containers which collect and manage personal physical, biological, and physiological data. Further, the HDT may incorporate and utilize the data from, for example, wearable sensors to provide real-time, dynamic monitoring, analysis, and synchronized representation of its twin, the real biological human. The HDT can be used in human-centered applications including personalized products and services, by furnishing specific data of an individual to a potential user of the data upon the request from the user and the authorization by the owner of the HDT.

Further, the disclosed system may include digital human models which describe particular aspects of human features or characteristics for a group of people, avatars which use computer graphics to virtually mimic certain human features (e.g., human shape, facial expression, and human motion), and human agents which incorporate human data into particular forms to represent particular attributes or behaviors.

Further, the disclosed method may facilitate the generation of the HDT. Further, the disclosed method may include steps of (1) template model creation; (2) model unification; (3) individualization; and (d) data collection and management.

1) Template model creation: To reduce the efforts required for generating the HDT and expedite the generating process, the state-of-the-art standard models may be built in advance and then used as the template models for the individualized models.

2) Model unification: Conventionally, human body shape (with anthropometry), muscular-skeleton, and full-body anatomical structure are described separately by three independent models of a different type. However, human features/characteristics are inter-dependent and interactive. In particular, human body shape, muscular-skeleton, and full-body anatomical structure all are about human physical features and characteristics. Therefore, these models, each of which describes specific aspects of human, are integrated into a framework with a unified model structure and data structure to share common data and to facilitate information exchange. The unified model structure and data structure remain the same for every individual. The framework utilizes common features (data) shared among different models and implement unification by using the methods of fitting, scaling, and morphing.

3) Individualization: Humans are the same as we share common structures and characteristics. Therefore, the disclosed system creates a unified model structure and data structure, and standard models (templates) that will be used for all humans. Humans are different as each is unique and different from others. Therefore, the disclosed system creates individualized models for each person so that personal uniqueness is well described. Creating a digital human model for each person requires extensive work. To keep the effort tractable and affordable, this invention uses standard models as the templates and then create individualized (instance) models from the template models via fitting, morphing, and scaling based on the personal data. Further, the personal data used associated with the disclosed system include but are not limited to 2D imagery, 3D body scan, motion capture data, magnetic resonance imaging (MRI) data, and computed tomography (CT) data.

4) Data collection and management: Some human features or status are not suitable to be represented by a model or need to be updated frequently or monitored in real-time. Therefore, in reference to FIG. 5, an HDT data management system is created to collect data from various sources which include but are not limited to personal medical records, personal medical test reports, and wearable sensors put on an individual. Further, the HDT data management system may transfer the data into respective data containers. Further, the HDT data management system may store and manage the data with a data server. The personal data associated with HDT can be categorized in terms of physical, biological, and physiological features/characteristics. The personal data associated with HDT can be categorized in terms of static or dynamic. The personal data associated with HDT can be categorized in terms of discrete or continuous (sequential). The personal data associated with HDT can be categorized in terms of 1-D, 2-D, or 3-D; and (e) images, videos, signals, digital records, and text records.

Referring now to figures, FIG. 1 is an illustration of an online platform 100 consistent with various embodiments of the present disclosure. By way of non-limiting example, the online platform 100 for provisioning digital data of an individual may be hosted on a centralized server 102, such as, for example, a cloud computing service. The centralized server 102 may communicate with other network entities, such as, for example, a mobile device 106 (such as a smartphone, a laptop, a tablet computer, etc.), other electronic devices 110 (such as desktop computers, server computers, etc.), databases 114, and sensors 116 over a communication network 104, such as, but not limited to, the Internet. Further, users of the online platform 100 may include relevant parties such as, but not limited to, end-users, administrators, service providers, service consumers, and so on. Accordingly, in some instances, electronic devices operated by the one or more relevant parties may be in communication with the platform.

A user 112, such as the one or more relevant parties, may access online platform 100 through a web-based software application or browser. The web-based software application may be embodied as, for example, but not be limited to, a website, a web application, a desktop application, and a mobile application compatible with a computing device 1400.

FIG. 2 is a block diagram of a system 200 for provisioning digital data of an individual, in accordance with some embodiments. The provisioning of digital data may include generating, updating and providing access to a digital twin under the same umbrella term, thus preserving unity of disclosure.

Further, the system 200 may include a communication device 202 configured for receiving a request from a business provider device associated with a business provider. Further, the request may include an identifier associated with the individual and at least one key associated with at least one data block of a digital twin data associated with the individual. Further, in some embodiments, the individual may include a human being. Further, the digital twin data may include structural data representing at least one structure of the individual, at least one functional data representing at least one function of the individual, and behavioral data representing at least one behavior of the individual. Further, the communication device 202 may be configured for transmitting the at least one data block of the digital twin data to the business provider device. Further, the business provider device may be configured to use the at least one data block to personalize at least one of a product and a service provisioned to the individual. Further, the system 200 may include a processing device 204 communicatively coupled to the communication device 202. Further, the processing device 204 may be configured for processing the request. Further, the processing device 204 may be configured for extracting the at least one data block based on the processing. This may include selectively extracting portions of the digital twin data and providing it to requesting parties. Further, the system 200 may include a storage device 206 communicatively coupled to the processing device 204. Further, the storage device 206 may be configured for retrieving the at least one data block.

Further, in some embodiments, the individual may include a non-human being, such as, for example, an animal, an artificially intelligent creature, a robotic system, and so on.

Further, in some embodiments, the structural data may include each of a three-dimensional human body shape model, a full-body musculoskeletal model, and a full-body anatomy model. Further, the at least one functional data may include physiological data.

Further, in some embodiments, the processing may include identifying the digital twin data amongst a plurality of digital twin data that may be included in the storage device 206 based on the identifier. Further, the extracting may include decrypting at least one encrypted data block based on the at least one key.

Further, in some embodiments, the processing device 204 may be configured for determining a template model data associated with a plurality of individuals. Further, the processing device 204 may be configured for individualizing the template model data based on personal data of the individual. Further, the processing device 204 may be configured for generating the digital twin data based on the individualizing. Further, the digital twin data may include a human digital twin data.

Further, in some embodiments, the individualizing may include performing at least one data transformation of the template model data. Further, the at least one data transformation may include at least one of fitting, morphing, and scaling.

Further, in some embodiments, the personal data may include at least one of a two-dimensional image of the individual, a three-dimensional image or scan of the individual, a motion capture data, a magnetic resonance imaging (MRI) data and a computed tomography (CT) data.

In further embodiments, the system 200 may include a plurality of sensors 302-304 (as shown in FIG. 3) communicatively coupled to the processing device 204. Further, the plurality of sensors 302-304 may be configured to generate sensor data corresponding to a plurality of characteristics of the individual. Further, the plurality of characteristics corresponds to at least two of the at least one structure, the at least one function and the at least one behavior. Further, the processing device 204 may be configured for analyzing the sensor data. Further, the processing device 204 may be configured for updating the digital twin data based on the analyzing of the sensor data.

Further, in some embodiments, the template model data may include a muscular-skeleton model, a full body anatomy model, and a body shape model. Further, the individualizing may include generating a first unified model using the muscular-skeleton model and the full body anatomy model based on identification of shared data of bone structure and joint centers as reference. Further, the at least one data transformation may include scaling. Further, the individualizing may include generating a second unified model using the full body anatomy model and the body shape model based on shared data of body surface as reference. Further, the at least one data transformation may include morphing of the second unified model. Further, the individualizing may include generating a third unified model using the body shape model and the muscular-skeleton model based on shared data of landmarks as reference. Further, the at least one data transformation may include fitting the third unified model.

Further, in some embodiments, the communication device 202 may be configured for receiving a plurality of additional data associated with the individual from a plurality of additional data sources. Further, the generating of the digital twin data may be based on the additional data. Further, the plurality of additional data may include personal medical records, personal medical test reports, and wearable sensors, and so on.

Further, in some embodiments, the template model data may include a muscular-skeleton model, a full body anatomy model, and a body shape model. Further, the individualizing may include performing at least one of reconstructing the body shape model from the two-dimensional image using deep learning and reconstructing the body shape model by fitting a template body shape model with the three-dimensional image. Further, the individualizing may include deriving each of the muscular-skeleton model and the full body anatomy model based on the body shape model.

Further, in some embodiments, the template model data may include a muscular-skeleton model, a full body anatomy model, and a body shape model. Further, the individualizing may include reconstructing the full body anatomy model by fitting a template model with the CT data. Further, the individualizing may include deriving each of the body shape model and the muscular-skeleton model based on the full body anatomy model.

In some embodiments, a computer server may be used by a HDT vendor. Therefore, the system 200 is characterized to include the communication device 202 (for communicating with client devices), the processing device 204 and the storage device 206.

FIG. 3 is a block diagram of the system 200 for provisioning the digital data of the individual, in accordance with some embodiments.

FIG. 4 is a flowchart of a method 400 for provisioning digital data of an individual, in accordance with some embodiments. Accordingly, at 402, the method 400 may include receiving, using a communication device, a request from a business provider device associated with a business provider. Further, the request may include an identifier associated with the individual and at least one key associated with at least one data block of a digital twin data associated with the individual. Further, the digital twin data may include structural data representing at least one structure of the individual, at least one functional data representing at least one function of the individual, and behavioral data representing at least one behavior of the individual.

Further, at 404, the method 400 may include processing, using a processing device, the request.

Further, at 406, the method 400 may include retrieving, using a storage device, the at least one data block.

Further, at 408, the method 400 may include extracting, using the processing device, the at least one data block based on the processing.

Further, at 410, the method 400 may include transmitting, using the communication device, the at least one data block of the digital twin data to the business provider device. Further, the business provider device may be configured to use the at least one data block to personalize at least one of a product and a service provisioned to the individual.

Further, in some embodiments, the individual may include a human being. In some embodiments, the individual may include a non-human being, such as, for example, an animal, an artificially intelligent creature, a robotic system, and so on.

Further, in some embodiments, the processing may include identifying the digital twin data amongst a plurality of digital twin data that may be included in the storage device based on the identifier. Further, the extracting may include decrypting at least one encrypted data block based on the at least one key.

Further, in some embodiments, the structural data comprises each of a three-dimensional human body shape model, a full-body musculoskeletal model, and a full-body anatomy model, wherein the at least one functional data comprises physiological data.

FIG. 5 is a flowchart of a method 500 for generating the digital twin data, in accordance with some embodiments. Further, at 502, the method 500 may include determining, using the processing device, a template model data associated with a plurality of individuals.

Further, at 504, the method 500 may include individualizing, using the processing device, the template model data based on personal data of the individual.

Further, at 506, the method 500 may include generating, using the processing device, the digital twin data based on the individualizing. Further, the digital twin data comprises a human digital twin data.

Further, in some embodiments, the individualizing may include performing at least one data transformation of the template model data. Further, the at least one data transformation may include at least one of fitting, morphing, and scaling.

Further, in some embodiments, the personal data may include at least one of a two-dimensional image of the individual, a three-dimensional image or scan of the individual, a motion capture data, a magnetic resonance imaging (MRI) data, and a computed tomography (CT) data.

In further embodiments, the method 500 may include receiving, using the communication device, a plurality of additional data associated with the individual from a plurality of additional data sources. Further, the generating of the digital twin data may be based on the additional data. Further, the plurality of additional data may include personal medical records, personal medical test reports, and data from wearable sensors, and so on.

FIG. 6 is a flowchart of a method 600 for updating the digital twin data, in accordance with some embodiments. Accordingly, at 602, the method 600 may include generating, using a plurality of sensors communicatively coupled to the processing device, sensor data corresponding to a plurality of characteristics of the individual. Further, the plurality of characteristics corresponds to at least two of the at least one structure, the at least one function, and the at least one behavior.

Further, at 604, the method 600 may include analyzing, using the processing device, the sensor data.

Further, at 606, the method 600 may include updating, using the processing device, the digital twin data based on the analyzing of the sensor data.

FIG. 7 is a flowchart of a method 700 for the individualizing the template model data, in accordance with some embodiments. Accordingly, the template model data may include a muscular-skeleton model, a full body anatomy model, and a body shape model. Further, at 702, the method 700 may include generating a first unified model using the muscular-skeleton model and the full body anatomy model based on identification of shared data of bone structure and joint centers as reference. Further, the at least one data transformation may include scaling of the first unified model.

Further, at 704, the method 700 may include generating a second unified model using the full body anatomy model and the body shape model based on shared data of body surface as reference. Further, the at least one data transformation may include morphing of the second unified model.

Further, at 706, the method 700 may include generating a third unified model using the body shape model and the muscular-skeleton model based on shared data of landmarks as reference. Further, the at least one data transformation may include fitting the third unified model.

FIG. 8 is a flowchart of a method 800 for individualizing the template model data, in accordance with some embodiments. Accordingly, the template model data may include a muscular-skeleton model, a full body anatomy model, and a body shape model. Further, at 802, the method 800 may include performing at least one of reconstructing the body shape model from the two-dimensional image using deep learning and reconstructing the body shape model by fitting a template body shape model with the three-dimensional image.

Further, at 804, the method 800 may include deriving each of the muscular-skeleton model and the full body anatomy model based on the body shape model.

FIG. 9 is a flowchart of a method 900 for individualizing the template model data, in accordance with some embodiments. Accordingly, the template model data may include a muscular-skeleton model, a full body anatomy model, and a body shape model. Further, at 902, the method 900 may include reconstructing the full body anatomy model by fitting a template model with the CT data.

Further, at 904, the method 900 may include deriving each of the body shape model and the muscular-skeleton model based on the full body anatomy model.

FIG. 10 is a flow diagram of a method 1000 for facilitating generating of a human digital twin, in accordance with some embodiments. Accordingly, the method 1000 may include a body shape model 1002. Further, the method 1000 may include a musculoskeletal model 1004. Further, the method 1000 may include a full body Finite Element (FE) model 1006. Further, the method 1000 may include other body models 1008. Further, the body shape mode 1002 may provide a complete 3D surface mesh description of human body shape along with anthropometric measurements. Further, the musculoskeletal model 1004 may include bony geometry, rigid linkage with multiple degrees of freedom to define joint kinematics, and Hill-type models of muscles and tendons and provides a non-invasive means to study human kinematics and movement. Further, the full body FE model (or full-body anatomy FE model) 1006 may use solid FE meshes to describe the complete anatomical structure of the human body in terms of tissue groups. Further, the other body models 1008 may include a human behavior model, a lifestyle model, and a cognitive model that may be integrated. Further, at 1010, the method 1000 may include unification of model structure (such as the body shape model 1002, the musculoskeletal model 1004, the full body FE model 1006, and other body models 1008) and data structure. Further, at 1012, the method 1000 may include a step of individualization. Further, the individualization may be performed using fitting, morphing, and scaling. Further, the method 1000 may include personal data 1018. Further, the personal data 1018 may include but are not limited to a 2D imagery, a 3D body scan, a motion capture data, a magnetic resonance imaging (MRI) data, and a computed tomography (CT) data. Further, the personal data 1018 may be categorized in terms of physical, biological, and physiological features/characteristics. Further, the personal data 1018 may be categorized in terms of static or dynamic. Further, the personal data 1018 may be categorized in terms of discrete or continuous (sequential). Further, the personal data 1018 may be categorized in terms of 1-D, 2-D, or 3-D. Further, the personal data 1018 may be categorized in terms of images, videos, signals, digital records, and text records. Further, the method 1000 may include individualized and unified digital human models 1014. Further, the individualized and unified digital human models 1014 may represent human anthropometric and biomechanical features and characteristics. Further, data containers associated with the individualized and unified digital human models 1014 may be used to store and manage personal physical, biological, and physiological data, health data, activity data, and medical data, and many other types of data. Further, at 1016, the method 1000 may include a step of generation of the human digital twin.

Further, if the personal data 1018 provided is 2D images and/or videos, a computer vision and deep learning-based method may be used to reconstruct a 3D shape model (such as the body shape model 1002) from 2D imagery. Further, the musculoskeletal model 1004 and the full body FE model 1006 may be derived from the body shape model 1002.

Further, if the personal data 1018 provided is a 3D body scan, a template body shape model may be fitted into the 3D body scan to get the body shape model 1002. Further, the musculoskeletal model 1004 and the full body anatomy FE model 1006 may be derived from the body shape model 1002.

Further, if the personal data 1018 provided is a CT scan, the full body anatomy FE model 1006 may be derived from the CT scan by fitting a template model to the CT scan. Further, the body shape model 1002 and the musculoskeletal model 1004 may be derived from the full body anatomy FE model 1006.

Further, a human body shape (with anthropometry), muscular-skeleton, and full body anatomical structure may be described separately by three independent models (such as the body shape model 1002, the musculoskeletal model 1004, and the full body FE model 1006) of a different type. However, human features/characteristics are inter-dependent and interactive. Further, the body shape model 1002, the musculoskeletal model 1004, and the full body FE model 1006 may be associated with human physical features and characteristics. Further, the body shape model 1002, the musculoskeletal model 1004, and the full body FE model 1006 describing specific aspects of human, may be integrated into a framework with a unified model structure (such as the individualized and unified digital human models 1014) and data structure to share common data and to facilitate information exchange. The unified model structure and data structure may remain the same for every individual.

FIG. 11 is a flow diagram of a method 1100 for facilitating the unification of model structures, in accordance with some embodiments. Accordingly, the model structures may include a body shape model 1102, a muscular-skeleton model 1104, and a full body anatomy Finite Element (FE) model. Further, at 1108, the method 1100 may include the unification of the muscular-skeleton model 1104 with the full body anatomy FE model 1106 using the shared data of bone structure and joint centers as the reference and may be achieved through scaling. Further, at 1110, the method 1100 may include unification of the full body anatomy FE model 1106 with the body shape model 1102 using shared data of body surface/skin as the reference and may be achieved through morphing. Further, at 1112, the method 1100 may include unification of the body shape model 1102 with the muscular-skeleton model 1104 using the shared data of landmarks as the reference and may be achieved through fitting.

FIG. 12 is a flow diagram of a Human Digital Twin (HDT) data management system 1200, in accordance with some embodiments. Accordingly, some human features or status may not be suitable to be represented by a model or need to be updated frequently or monitored in real-time. Further, the HDT data management system 1200 (such as the system 200) may be configured for receiving data from various sources that may include, but are not limited to, medical records 1202 (or personal medical records), medical test reports 1204 (or personal medical test reports), and wearable sensors 1206 put on an individual. Further, at 1208, the HDT data management system 1200 may be configured for transmitting the data into data containers such as physical data 1210, biological data 1212, and physiological data 1214. Further, the HDT data management system 1200 may be configured for storing and managing the data to a data server 1216.

FIG. 13 is a block diagram showing an HDT vendor 1302, a human individual 1304, a business 1306, and a Human Digital Twin (HDT) 1308, in accordance with some embodiments. Accordingly, the HDT vendor 1302 may build, maintain, and update the HDT 1308 for the human individual 1304. Further, the human individual 1304 may be a customer to the HDT vendor 1302. Further, the human individual 1304 may own the HDT 1308 and control the access and use of the data contained. When the business 1306 intends to provide individualized or personalized products/services to the human individual 1304, the business 1306 may request the human individual 1304, that may be a customer to the business 1306, to authorize the access and use of a specific data block from the HDT 1308 that may be required for the products/services. Further, the business 1306 may provide furnishing tools for the human individual 1304 to use the Human Digital Twin (HDT) 1308. If the human individual 1304 approves the request, the human individual 1304 may provide a unique key to the business 1306 for the specific data block. Further, the business 1306 may submit the key to the HDT vendor 1302 to get the specific data block requested.

With reference to FIG. 14, a system consistent with an embodiment of the disclosure may include a computing device or cloud service, such as computing device 1400. In a basic configuration, computing device 1400 may include at least one processing unit 1402 and a system memory 1404. Depending on the configuration and type of computing device, system memory 1404 may comprise, but is not limited to, volatile (e.g. random-access memory (RAM)), non-volatile (e.g. read-only memory (ROM)), flash memory, or any combination. System memory 1404 may include operating system 1405, one or more programming modules 1406, and may include a program data 1407. Operating system 1405, for example, may be suitable for controlling computing device 1400's operation. In one embodiment, programming modules 1406 may include image-processing module, machine learning module. Furthermore, embodiments of the disclosure may be practiced in conjunction with a graphics library, other operating systems, or any other application program and is not limited to any particular application or system. This basic configuration is illustrated in FIG. 14 by those components within a dashed line 1408.

Computing device 1400 may have additional features or functionality. For example, computing device 1400 may also include additional data storage devices (removable and/or non-removable) such as, for example, magnetic disks, optical disks, or tape. Such additional storage is illustrated in FIG. 14 by a removable storage 1409 and a non-removable storage 1410. Computer storage media may include volatile and non-volatile, removable and non-removable media implemented in any method or technology for storage of information, such as computer-readable instructions, data structures, program modules, or other data. System memory 1404, removable storage 1409, and non-removable storage 1410 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 1400. Any such computer storage media may be part of device 1400. Computing device 1400 may also have input device(s) 1412 such as a keyboard, a mouse, a pen, a sound input device, a touch input device, a location sensor, a camera, a biometric sensor, etc. Output device(s) 1414 such as a display, speakers, a printer, etc. may also be included. The aforementioned devices are examples and others may be used.

Computing device 1400 may also contain a communication connection 1416 that may allow device 1400 to communicate with other computing devices 1418, such as over a network in a distributed computing environment, for example, an intranet or the Internet. Communication connection 1416 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 1404, including operating system 1405. While executing on processing unit 1402, programming modules 1406 (e.g., application 1420) may perform processes including, for example, one or more stages of methods, algorithms, systems, applications, servers, databases as described above. The aforementioned process is an example, and processing unit 1402 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

Although the present disclosure has been explained in relation to its preferred embodiment, it is to be understood that many other possible modifications and variations can be made without departing from the spirit and scope of the disclosure.

What is claimed is:

1. A system for provisioning digital data of an individual, the system comprising:
a communication device configured for:
receiving a request from a business provider device associated with a business provider, wherein the request comprises an identifier associated with the individual and at least one key associated with at least one data block of a digital twin data associated with the individual, wherein the digital twin data comprises structural data representing at least one structure of the individual, at least one functional data representing at least one function of the individual and behavioral data representing at least one behavior of the individual;
transmitting the at least one data block of the digital twin data to the business provider device, wherein the business provider device is configured to use the at least one data block to personalize at least one of a product and a service provisioned to the individual;
a processing device communicatively coupled to the communication device, wherein the processing device is configured for:
processing the request; and
extracting the at least one data block based on the processing; and
a storage device communicatively coupled to the processing device, wherein the storage device is configured for retrieving the at least one data block.

2. The system of claim 1, wherein the processing comprises identifying the digital twin data amongst a plurality of digital twin data comprised in the storage device based on the identifier, wherein the extracting comprises decrypting at least one encrypted data block based on the at least one key.

3. The system of claim 1, wherein the processing device is further configured for:
determining a template model data associated with a plurality of individuals;
individualizing the template model data based on personal data of the individual; and
generating the digital twin data based on the individualizing, wherein the digital twin data comprises a human digital twin data.

4. The system of claim 3, wherein the individualizing comprises performing at least one data transformation of the template model data, wherein the at least one data transformation comprises at least one of fitting, morphing and scaling.

5. The system of claim 4, wherein the personal data comprises at least one of a two-dimensional image of the individual, a three-dimensional image or scan of the individual, a motion capture data, a magnetic resonance imaging (MRI) data and a computed tomography (CT) data.

6. The system of claim 3 further comprising:
a plurality of sensors communicatively coupled to the processing device, wherein the plurality of sensors is configured to generate sensor data corresponding to a plurality of characteristics of the individual, wherein the plurality of characteristics corresponds to at least two of the at least one structure, the at least one function and the at least one behavior, wherein the processing device is further configured for:
analyzing the sensor data; and
updating the digital twin data based on the analyzing of the sensor data.

7. The system of claim 3, wherein the template model data comprises a muscular-skeleton model, a full body anatomy model and a body shape model, wherein the individualizing further comprises:
generating a first unified model using the muscular-skeleton model and the full body anatomy model based on identification of shared data of bone structure and joint centers as reference, wherein the at least one data transformation comprises scaling of the first unified model;
generating a second unified model using the full body anatomy model and the body shape model based on shared data of body surface as reference, wherein the at least one data transformation comprises morphing of the second unified model; and
generating a third unified model using the body shape model and the muscular-skeleton model based on shared data of landmarks as reference, wherein the at least one data transformation comprises fitting the third unified model.

8. The system of claim 3, wherein the communication device is further configured for receiving a plurality of additional data associated with the individual from a plurality of additional data sources, wherein the generating of the digital twin data is based further on the additional data.

9. The system of claim 5, wherein the template model data comprises a muscular-skeleton model, a full body anatomy model and a body shape model, wherein the individualizing comprises:
performing at least one of:
reconstructing the body shape model from the two-dimensional image using deep learning; and
reconstructing the body shape model by fitting a template body shape model with the three-dimensional image or scan; and
deriving each of the muscular-skeleton model and the full body anatomy model based on the body shape model.

10. The system of claim 5, wherein the template model data comprises a muscular-skeleton model, a full body anatomy model and a body shape model, wherein the individualizing comprises:
reconstructing the full body anatomy model by fitting a template model with the CT data; and
deriving each of the body shape model and the muscular-skeleton model based on the full body anatomy model.

11. A method for provisioning digital data of an individual, the method comprising:
receiving, using a communication device, a request from a business provider device associated with a business provider, wherein the request comprises an identifier associated with the individual and at least one key associated with at least one data block of a digital twin data associated with the individual, wherein the digital twin data comprises structural data representing at least one structure of the individual, at least one functional data representing at least one function of the individual and behavioral data representing at least one behavior of the individual;
processing, using a processing device, the request;
retrieving, using a storage device, the at least one data block;
extracting, using the processing device, the at least one data block based on the processing; and
transmitting, using the communication device, the at least one data block of the digital twin data to the business provider device, wherein the business provider device is configured to use the at least one data block to personalize at least one of a product and a service provisioned to the individual.

12. The method of claim 11, wherein the processing comprises identifying the digital twin data amongst a plurality of digital twin data comprised in the storage device based on the identifier, wherein the extracting comprises decrypting at least one encrypted data block based on the at least one key.

13. The method of claim 11 further comprising:
determining, using the processing device, a template model data associated with a plurality of individuals;
individualizing, using the processing device, the template model data based on personal data of the individual; and
generating, using the processing device, the digital twin data based on the individualizing, wherein the digital twin data comprises a human digital twin data.

14. The method of claim 13, wherein the individualizing comprises performing at least one data transformation of the template model data, wherein the at least one data transformation comprises at least one of fitting, morphisng and scaling.

15. The method of claim 14, wherein the personal data comprises at least one of a two-dimensional image of the individual, a three-dimensional image of the individual, a motion capture data, a magnetic resonance imaging (MRI) data and a computed tomography (CT) data.

16. The method of claim 13 further comprising:
generating, using a plurality of sensors communicatively coupled to the processing device, sensor data corresponding to a plurality of characteristics of the individual, wherein the plurality of characteristics corresponds to at least two of the at least one structure, the at least one function and the at least one behavior;
analyzing, using the processing device, the sensor data; and
updating, using the processing device, the digital twin data based on the analyzing of the sensor data.

17. The method of claim 13, wherein the template model data comprises a muscular-skeleton model, a full body anatomy model and a body shape model, wherein the individualizing further comprises:
generating a first unified model using the muscular-skeleton model and the full body anatomy model based on identification of shared data of bone structure and joint centers as reference, wherein the at least one data transformation comprises scaling of the first unified model;
generating a second unified model using the full body anatomy model and the body shape model based on shared data of body surface as reference, wherein the at least one data transformation comprises morphing of the second unified model; and
generating a third unified model using the body shape model and the muscular-skeleton model based on shared data of landmarks as reference, wherein the at least one data transformation comprises fitting the third unified model.

18. The method of claim 13 further comprising receiving, using the communication device, a plurality of additional data associated with the individual from a plurality of additional data sources, wherein the generating of the digital twin data is based further on the additional data.

19. The method of claim 15, wherein the template model data comprises a muscular-skeleton model, a full body anatomy model and a body shape model, wherein the individualizing comprises:
performing at least one of:
reconstructing the body shape model from the two-dimensional image using deep learning; and
reconstructing the body shape model by fitting a template body shape model with the three-dimensional image or scan; and
deriving each of the muscular-skeleton model and the full body anatomy model based on the body shape model.

20. The method of claim 15, wherein the template model data comprises a muscular-skeleton model, a full body anatomy model and a body shape model, wherein the individualizing comprises:
reconstructing the full body anatomy model by fitting a template model with the CT data; and
deriving each of the body shape model and the muscular-skeleton model based on the full body anatomy model.
